(19) United States
(12) Patent Application Publication
(10) Pub. No.: US 2014/0063454 A1
(43) Pub. Date: Mar. 6, 2014

(54) OPHTHALMOLOGIC IMAGING APPARATUS AND OPHTHALMOLOGIC IMAGING METHOD

(71) Applicant: Canon Kabushiki Kaisha, Tokyo (JP)

(72) Inventor: Shohhei Kamada, Tokyo (JP)

(21) Appl. No.: 14/013,463

(22) Filed: Aug. 29, 2013

(30) Foreign Application Priority Data
Sep. 5, 2012 (JP) 2012-195299

(51) Int. Cl. A61B 3/14

(52) U.S. Cl. CPC A61B 3/14
USPC 351/206

(57) ABSTRACT

Provided is an ophthalmologic imaging apparatus, including: an imaging light source for illuminating a fundus; a light intensity detection unit for monitoring, at a time of imaging, an emission intensity from a start of emission of the imaging light source; an imaging light wavelength selection unit for selecting a wavelength band of light that is emitted from the imaging light source and illuminates the fundus; and a detection wavelength changing unit for changing a wavelength band of light to be guided from the imaging light source to the light intensity detection unit depending on the wavelength band selected by the imaging light wavelength selection unit.

### BACKGROUND OF THE INVENTION

1. Field of the Invention

The present invention relates to an ophthalmologic imaging apparatus that is configured to control an imaging light intensity at the time of imaging an eye to be inspected.

2. Description of the Related Art

In a conventional ophthalmologic apparatus such as a fundus camera, a light source such as a xenon tube is used as a light source for imaging a fundus of an eye to be inspected. The light source generally changes in emission intensity due to degradation caused by a change with time. Therefore, a part of return light from the eye to be inspected is monitored by a light receiving element such as a photodiode so as to obtain a constant imaging light intensity. An output from the light receiving element is integrated by an integrating circuit including an operational amplifier and a capacitor, and an output from the integrating circuit is compared with a standard voltage. When the output becomes the standard voltage or more, emission is stopped to perform control so that the imaging light intensity has a constant value, to thereby obtain a fundus image having equal lightness at all times (see Japanese Patent Application Laid-Open No. S60-190930).

The conventional fundus camera also has a plurality of imaging modes, and an operator may change the imaging mode depending on the imaging purpose. For example, there have been known a color imaging mode in which the fundus is illuminated with white light to acquire an image, a filter imaging mode in which the fundus is illuminated with light having a narrow-band wavelength of around 500 nm to acquire an image so that a nerve fiber layer of the fundus may be observed more clearly, and the like. Such plurality of imaging modes is realized by employing a configuration in which a filter is placed to be removably insertable on an optical axis between the imaging light source and the eye to be inspected, and inserting and removing the filter to selectively extract a partial wavelength of a wavelength band of the imaging light source.

In the fundus camera in which the plurality of imaging modes is realized by interposing the filter and the like between the imaging light source and the fundus, the light emitted by the imaging light source and light that actually illuminates the fundus have different wavelength distributions. In general, imaging light sources have individually different emission wavelength distributions, and hence in a method in which the emission of the imaging light source is directly received and imaging light is controlled based on the output, the difference in wavelength distribution between the light emitted by the imaging light source, that is, the monitored light, and the light that actually illuminates the fundus leads to a control error.

As described above, in the ophthalmologic apparatus having the configuration exemplified in Japanese Patent Application Laid-Open No. S60-190930, the reflected light from the eye to be inspected is monitored to control the imaging light so that the control error due to the difference in wavelength distribution may be reduced.

However, the reflected light from the eye to be inspected generally contains various ghosts such as flare due to misalignment. Therefore, in the case where the imaging light is controlled based on the reflected light, although a desired average brightness of the acquired image may be obtained, problems such as improper exposure of a fundus part, which is required for diagnosis, may occur due to effects of the ghosts contained in the reflected light.

### SUMMARY OF THE INVENTION

The present invention has been made in view of the above-mentioned circumstances, and therefore has an object to provide an ophthalmologic imaging apparatus capable of reducing a control error due to a difference in wavelength distribution and adjusting a light intensity while reducing effects of ghosts and the like contained in reflected light.

An ophthalmologic imaging apparatus according to one embodiment of the present invention includes:
an imaging light source for illuminating a fundus;
a light intensity detection unit for monitoring, at a time of imaging, an emission intensity from a start of emission of the imaging light source;
an imaging light wavelength selection unit for selecting a wavelength band of light that is emitted from the imaging light source and illuminates the fundus; and
a detection wavelength changing unit for changing a wavelength band of light to be guided from the imaging light source to the light intensity detection unit depending on the wavelength band selected by the imaging light wavelength selection unit.

According to one embodiment of the present invention, the control error due to the difference in wavelength distribution may be reduced and the light intensity may be adjusted while reducing the effects of ghosts and the like contained in the reflected light.

Further features of the present invention will become apparent from the following description of exemplary embodiments with reference to the attached drawings.

### BRIEF DESCRIPTION OF THE DRAWINGS

FIG. 1 is a configuration diagram of a fundus camera according to a first embodiment of the present invention.

FIG. 2 is a graph showing wavelengths of imaging light and spectral sensitivity characteristics of photodiodes according to the first embodiment.

FIG. 3 is an electrical circuit diagram of a xenon tube drive circuit and a light intensity detection unit according to the first embodiment.

FIG. 4 is a flowchart of imaging according to the first embodiment.

FIGS. 5A and 5B are timing charts from start to stop of emission according to the first embodiment, of which FIG. 5A illustrates a timing chart for color imaging, and FIG. 5B illustrates a timing chart for filter imaging.

FIG. 6 is a configuration diagram of a fundus camera according to a second embodiment of the present invention.

FIG. 7 is an electrical circuit diagram of a xenon tube drive circuit and a light intensity detection unit according to the second embodiment.

FIG. 8 is a flowchart of imaging according to the second embodiment.

FIG. 9 is a configuration diagram of a fundus camera according to a third embodiment of the present invention.

FIG. 10 is a flowchart of imaging according to the third embodiment.

### DESCRIPTION OF THE EMBODIMENTS

### First Embodiment

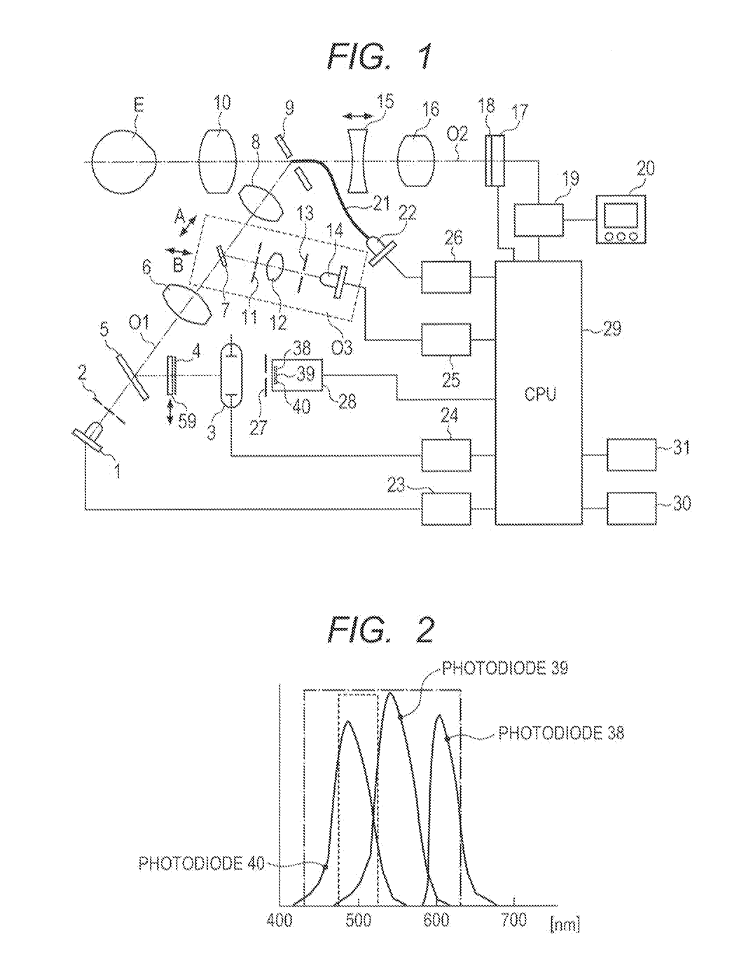

The present invention is described in detail by way of a first embodiment illustrated in FIGS. 1 to 5B.

FIG. 1 is a configuration diagram of a fundus camera according to the first embodiment. In an optical path from an objective lens 10 to a xenon tube 3 as a light source for imaging, which emits visible light, there are arranged a perforated mirror 9, a relay lens 8, a mirror 7, and a relay lens 6, and then the optical path leads to a dichroic mirror 5 for transmitting infrared light and reflecting the visible light. In a transmission direction of the dichroic mirror 5, a stop 2 having a ring-shaped aperture, and then an infrared LED 1 as a light source for infrared observation are arranged. In a reflection direction of the dichroic mirror 5, a stop 4 having a ring-shaped aperture, and then the xenon tube 3 are arranged to constitute a fundus illumination optical system 01. A band pass filter 59 for filter imaging is arranged between the stop 4 and the dichroic mirror 5 so as to be retractable out of an optical axis by a drive system (not shown) and retracted out of the optical axis during color imaging.

In a reflection direction of the mirror 7, an aperture 11, a lens 12, a focusing index 13, and an infrared LED 14 as a focusing index light source are arranged to constitute a focusing-index projection optical system O3.

The focusing-index projection optical system O3 is configured to move in a direction A in FIG. 1 in conjunction with a focusing lens 15. When acquiring a static image, the focusing-index projection optical system 03 moves in a direction B in FIG. 1 by the drive system (not shown) to be retracted out of the fundus illumination optical system O1.

In an optical path in a transmission direction of the perforated mirror 9, the focusing lens 15, an imaging lens 16, and a CMOS sensor 17 are arranged to constitute a fundus imaging optical system O2. An output of the CMOS sensor 17 is sequentially connected to an image signal processing portion 19 and a display portion 20. An infrared LED 22 as an alignment index light source is connected to the perforated mirror 9 through an optical fiber 21.

Behind the xenon tube 3, a light intensity detection unit 28 including a photodiode (Red) 38, a photodiode (Green) 39, and a photodiode (Blue) 40 is arranged, and each of the photodiodes may receive a part of the light beams emitted from the xenon tube 3 through a stop 27. Each of the photodiodes of the light intensity detection unit 28 functions as a light intensity detection portion for monitoring an emission intensity from the start of the emission of the imaging light source during the imaging in the present invention.

The infrared LED 1 is connected to an LED drive circuit 23, the xenon tube 3 for imaging is connected to a xenon tube drive circuit 24, the infrared LED 14 is connected to an LED drive circuit 25, and the infrared LED 22 is connected to an LED drive circuit 26. The LED drive circuit 23, the xenon tube drive circuit 24, the LED drive circuit 25, the LED drive circuit 26, the light intensity detection unit 28, the CMOS sensor 17, the image signal processing portion 19, an operation portion 30, and a recording portion 31 are connected to a central processing unit (CPU) 29.

Further, a filter 18 is arranged on the CMOS sensor 17, in which three colors of red (R), green (G), and blue (B) are arranged in a mosaic shape on respective pixels of the CMOS sensor 17. An R filter can transmit light in the range of from the red light to the infrared light.

When observing the infrared light, the image signal processing portion 19 generates monochrome movie data by using an output of the R pixel, and outputs the movie to the display portion 20. On the other hand, when acquiring a color static image and a filter static image, the image signal processing portion 19 generates a color static image by using outputs of the R, G, and B pixels, and records the generated static image in the recording portion 31 via the CPU 29.

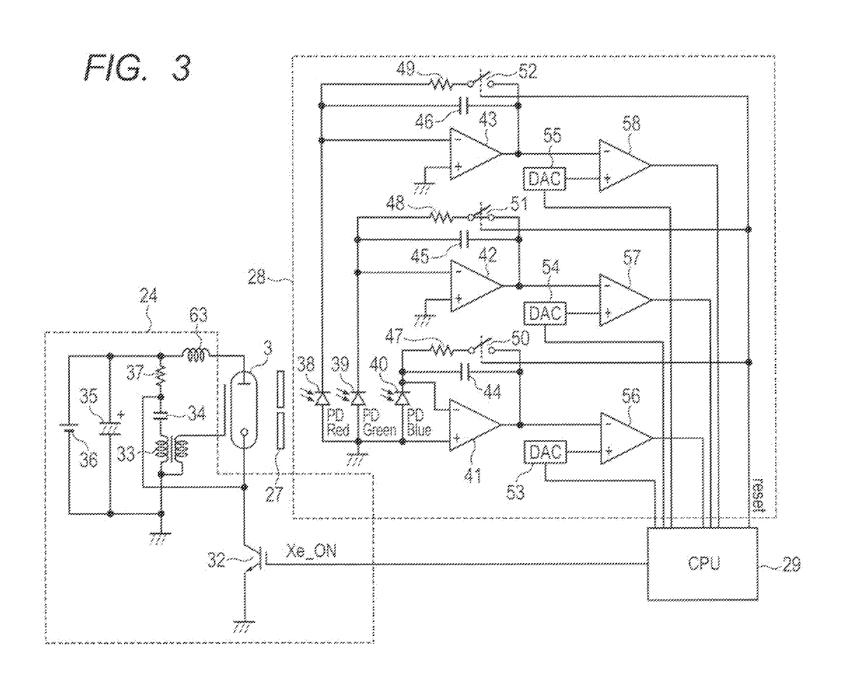

FIG. 2 is a graph showing wavelength bands of imaging light that illuminates an eye to be inspected during the imaging, and spectral sensitivity characteristics of the photodiodes according to this embodiment. At the time of color imaging, a fundus is illuminated with light in a wide band (range illustrated by the alternate long and short dash line) of about 430 to 630 nm, which is an illumination wavelength band of the xenon tube 3. Moreover, in both the color imaging and the filter imaging, the photodiode (Red) 38, the photodiode (Green) 39, and the photodiode (Blue) 40 receive the light in the wide band. At the time of filter imaging, the fundus is illuminated with light in a narrow band of about 475 to 525 nm, which is a pass band (range illustrated by the dotted line) of the band pass filter 59. The photodiode (Red) 38, the photodiode (Green) 39, and the photodiode (Blue) 40 have sensitivities in wavelength bands of about 580 to 680 nm, about 475 to 615 nm, and about 420 to 560 nm, respectively.

At the time of imaging, the light intensity detection unit 28 passes outputs of the photodiodes to the CPU 29. At the time of color imaging, the CPU 29 controls the xenon tube drive circuit 24 based on the outputs of all the photodiodes. At the time of filter imaging, the CPU 29 controls the xenon tube drive circuit 24 based only on a light intensity detection result obtained from the photodiode (Blue) 40.

FIG. 3 is a configuration diagram of electrical circuits of the xenon tube drive circuit 24 and the light intensity detection unit 28 according to this embodiment. The xenon tube drive circuit 24 includes an insulated gate bipolar transistor (IGBT) 32, a main capacitor 35, a power source 36, a resistor 37, a trigger capacitor 34, a trigger transformer 33, and a choke coil 63, and the main capacitor 35 is charged to a high voltage (for example, 300 volts) by the power source 36. The trigger capacitor 34 is also charged through the resistor 37. With this circuit configuration, when the CPU 29 sets a Xe_ON signal to Hi, the IGBT 32 is turned on, and the trigger capacitor 34 is discharged first so that a current flows to a first winding of the trigger transformer 33. This generates a high voltage on a second winding of the trigger transformer 33, and the xenon tube 3 is triggered. As a result, a current is allowed to flow from the main capacitor 35 to the xenon tube 3 via the choke coil 63, and an emission of the xenon tube 3 is started. After the emission is started, when the CPU 29 sets the Xe_ON signal to Low, the IGBT 32 is turned off and the current to the xenon tube 3 is blocked, so that the emission is stopped.

The light intensity detection unit 28 includes three circuits having the same configuration. First, a configuration of one of the three circuits is taken as an example for description. The light intensity detection unit 28 includes an integrating circuit including the photodiode (Red) 38, an integrating capacitor 46, a reset resistor 49, an analog switch 52, and an operational amplifier 43. When the CPU 29 turns on the analog switch 52, charges of the integrating capacitor 46 can be reset through the reset resistor 49. A digital-to-analog (D/A) converter 55 outputs a standard voltage for stopping the emission of the xenon tube 3. An output of the D/A converter 55 is connected to an input of a comparator 58 together with an output of the operational amplifier 43, so that an output voltage of the integrating circuit and an output voltage of the D/A converter 55 can be compared with each other. An output of the comparator 58 is connected to the CPU 29. When the output voltage of the integrating circuit is lower than the output voltage of the D/A converter 55, a Hi signal is output from the comparator 53, and in a reverse case, a Lo signal is output.

In the same manner, a combination of the photodiode (Green) 39, an integrating capacitor 45, a reset resistor 48, an analog switch 51, an operational amplifier 42, a D/A converter 54, and a comparator 57 constitutes a similar circuit. In the same manner, a combination of the photodiode (Blue) 40, an integrating capacitor 44, a reset resistor 47, an analog switch 50, an operational amplifier 41, a D/A converter 53, and a comparator 56 also constitutes a circuit.

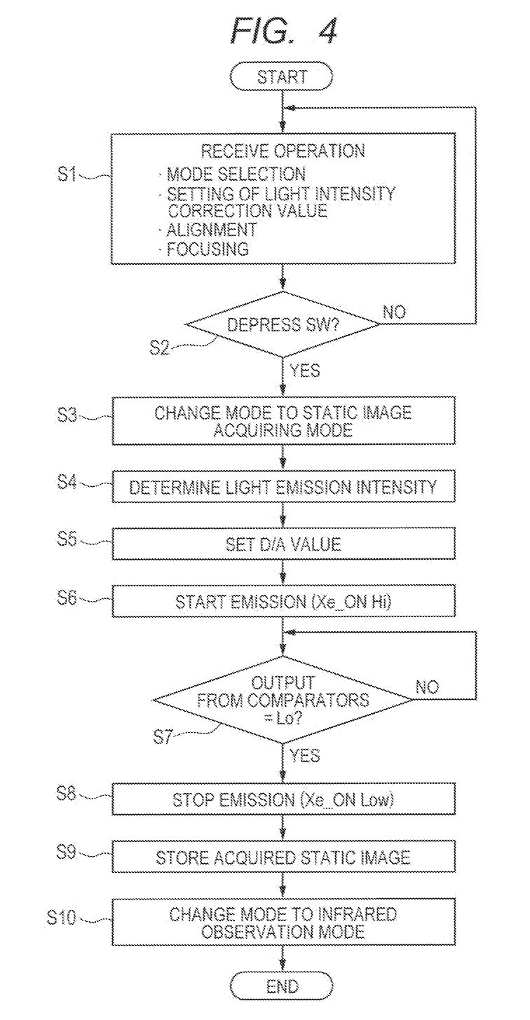

Now, a sequence from start to end of the imaging is described with reference to a flowchart of FIG. 4.

In Step S1, the fundus camera receives an operation by an operator. The operator operates a mode SW (not shown) of the operation portion 30 to select the color imaging or the filter imaging. When the color imaging is selected, the band pass filter 59 for the filter imaging is retracted from the optical axis. The operator also operates a light intensity adjustment SW (not shown) of the operation portion 30 to set a light intensity correction value at the time of imaging. The operator also performs alignment between the fundus camera and an eye to be inspected E with a fundus image of the eye to be inspected E illuminated by the infrared LED 1 as the light source for infrared observation, which is displayed on the display portion 20, and an alignment index image projected on a cornea of the eye to be inspected E by the infrared LED 22 as the alignment index light source. The operator also performs focusing with an index image of the infrared LED as the focusing index light source. The band pass filter 59 described above functions as an imaging light wavelength selection unit for selecting a predetermined wavelength band from the light that is emitted from the imaging light source and illuminates the fundus in the present invention. Note that, this embodiment employs a mode in which a specific wavelength band is extracted by the band pass filter 59, but as long as the wavelength of the fundus illumination light may be changed, various modes of changing the wavelength band, such as providing a plurality of the light sources to be used sequentially, may be employed.

When the alignment and the focusing are complete, the operator depresses an imaging SW (not shown) of the operation portion 30 to start the imaging (Step S2). At the time when the processing proceeds to Step S3, the analog switches 50, 51, and 52 of the light intensity detection unit 28 are in ON states, and the integrating capacitors 44, 45, and 46 are in reset states.

In Step S3, in order to change the mode from an infrared observation mode to a static image acquiring mode, the CPU 29 turns off the infrared LED 1, the infrared LED 22, and the infrared LED 14 and retracts the focusing index projection optical system O3 from the optical axis of the fundus illumination optical system O1.

In Step S4, a light emission intensity is calculated based on the set imaging mode and light intensity correction value.

In Step S5, based on the determined light emission intensity, a standard D/A value is determined with a predefined light intensity table, and the CPU 29 passes the standard D/A value to the D/A converters 53, 54, and 55. In other words, the photodiodes monitor the emission intensity of the imaging light source based on a standard signal depending on the emission intensity corresponding to the wavelength band of the light for illuminating the fundus, which is selected by the imaging light wavelength selection unit. It should be noted, however, that in the filter imaging, the control based on the outputs of the photodiode (Red) 38 and the photodiode (Green) 39 is not performed, and hence the standard D/A value is set to the maximum. In this manner, outputs of the comparators 58 and 57 become a normally Hi state. In other words, the CPU 29 as a control unit controls ones of the plurality of photodiodes that are not selected by a detection wavelength changing unit to output a predetermined signal corresponding to the Hi state in monitoring the light emitted from the xenon tube 3.

The D/A converter 55 outputs, based on the standard D/A value, a standard voltage Vrr to be compared with an output of the integrating circuit including the photodiode (Red) 38 to the comparator 58. In the same manner, the D/A converter 54 outputs a standard voltage Vrg to be compared with an output of the integrating circuit including the photodiode (Green) 39 to the comparator 57. In the same manner, the D/A converter 53 outputs a standard voltage Vrb to be compared with an output of the integrating circuit including the photodiode (Blue) 40 to the comparator 56. In other words, the CPU 29 functions as the detection wavelength changing unit for selecting, based on the wavelength band of the selected light to be used for illuminating the fundus, a wavelength band of light to be guided from the imaging light source to the light intensity detection unit. To be more specific, the CPU 29 switches one(s) of the plurality of photodiodes having different detection wavelengths for use depending on the selected wavelength band.

In Step S6, the CPU 29 turns off the analog switches 50, 51, and 52 to cancel the resetting of the integrating circuits. Thereafter, the CPU 29 sets the Xe_ON signal Hi to turn on the IGBT 32 and thereby trigger the xenon tube 3, which starts emission.

In Step S7, in the case of the color imaging, the CPU 29 first waits until the outputs of all the comparators 56, 57, and 58 become Lo, and when the outputs become Lo, the processing proceeds to Step S8. In the case of the filter imaging, the CPU 29 waits until the output of the comparator 56 for receiving the output of the integrating circuit including the photodiode (Blue) 40 becomes Lo, and when the output becomes Lo, the processing proceeds to Step S8.

In Step S8, the CPU 29 sets the Xe_ON signal Lo to turn off the IGBT 32 and thereby stop the emission of the xenon tube 3.

In Step S9, which is performed after the emission is stopped, the image signal processing portion 19 generates a static image corresponding to the imaging mode based on the output of the CMOS sensor 17 to be stored in the recording portion 31.

In Step S10, the mode is changed to the infrared observation mode.

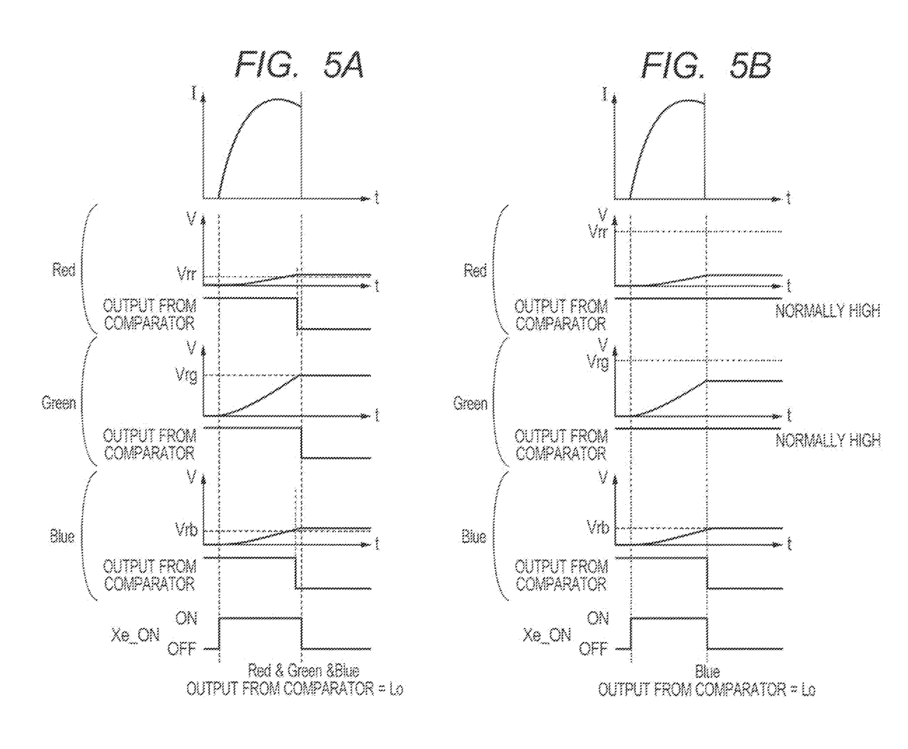

Now, the operation from the emission of Step S6 to the stop of the emission of Step S8 is described by way of an example of timing charts of FIGS. 5A and 5B. FIG. 5A illustrates a timing chart for the color imaging, and FIG. 5B illustrates a timing chart for the filter imaging.

In both of FIGS. 5A and 5B, the graph at the top shows the emission intensity of the xenon tube 3. The graphs at the middle respectively show, for the photodiode (Red) 38, the photodiode (Green) 39, and the photodiode (Blue) 40, the output voltages of the integrating circuits including the photodiodes and the outputs of the comparators for receiving the output voltages. The graph at the bottom shows the Xe_ON signal output by the CPU 29.

For the color imaging illustrated in FIG. 5A, the standard voltages are first set for Red, Green, and Blue. When the Xe_ON signal becomes Hi, the xenon tube 3 starts the emission. After the emission, when the outputs of the integrating circuits exceed the standard voltages, the comparators output Lo. At the moment when the outputs of all the comparators become Lo, the CPU 29 sets the Xe_ON signal Lo to turn off the IGBT 32 and thereby stop the emission of the xenon tube 3.

For the filter imaging illustrated in FIG. 5B, the standard voltages of Red and Green, which are not used for controlling the emission, are first set to the maximum, and the comparators output normally Hi. As in the color imaging, after the xenon tube 3 starts the emission, when the output of the integrating circuit including the photodiode (Blue) 40 exceeds the standard voltage, the comparator 56 outputs Lo. At that moment, the CPU 29 performs the control to stop the emission.

As described above, in this embodiment, the photodiodes as the light intensity detection portions directly monitor the light emitted from the xenon tube 3 without the emitted light passing through the fundus, and stop the emission of the xenon tube 3 depending on the monitoring result. Note that, as described later, various modifications may be made as long as the light does not pass the fundus.

According to the present invention, the wavelength to be monitored is thus switched to the wavelength band equivalent to the light that actually illuminates the fundus depending on the imaging mode so that a control error of the imaging light due to the individual difference of the imaging light sources in emission wavelength distribution may also be suppressed. Moreover, the control does not use the reflected light, and hence is not affected by the noise contained in the reflected light. In each of the imaging modes, the light for illuminating the fundus may be controlled to have a desired light intensity at high accuracy.

Moreover, the light source undergoes a change with time such as degradation with time depending on the frequency of use. However, as in the present invention, the control error of the light for use may be suppressed based on the light emitted by the light source at all times, and the effects of the change with time may also be suppressed.

### Second Embodiment

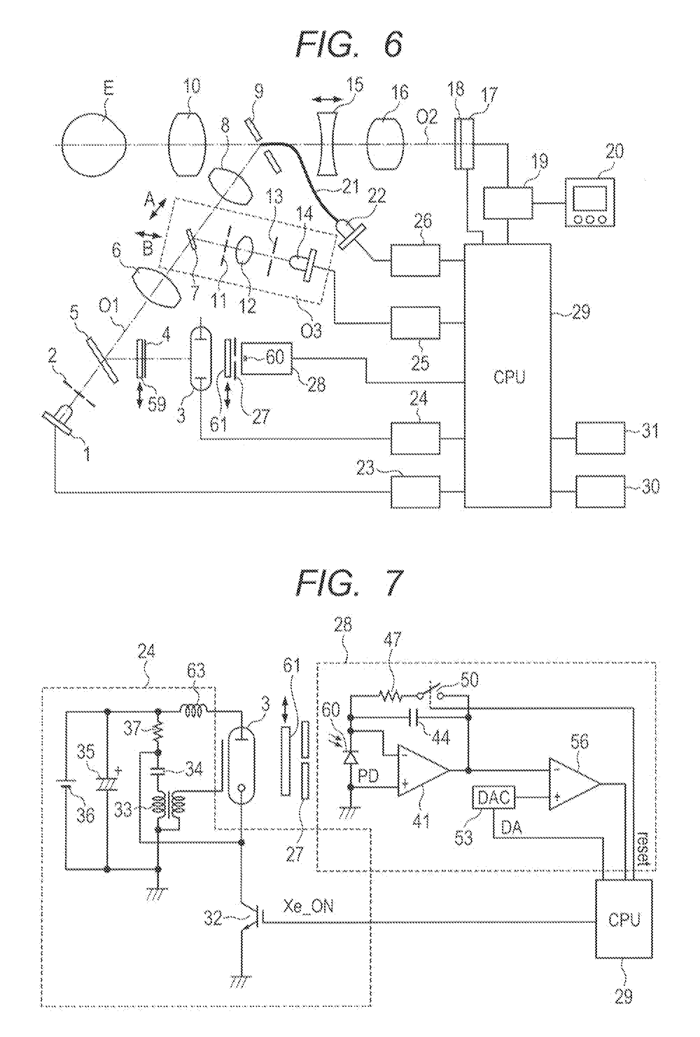

The present invention is described by way of a second embodiment illustrated in FIGS. 6 to 8.

The detailed description is given only of differences from the first embodiment.

FIG. 6 is a configuration diagram of a fundus camera according to the second embodiment. As a difference from the configuration of the first embodiment, a band pass filter 61 having the same spectral characteristics as the band pass filter 59 for the filter imaging is additionally placed between the xenon tube 3 and the stop 27. The band pass filter 61 is retractable out of the optical axis by a drive system (not shown) and retracted out of the optical axis during the color imaging. The band pass filter 61 functions as a band pass filter for detection unit provided between the imaging light source and the light intensity detection unit in the present invention. Moreover, depending on the band selected by the CPU 29 as the detection wavelength changing unit, the band pass filter 61 is inserted to and removed from the optical axis between the xenon tube 3 and the photodiodes 38, 39, and 40.

In addition, as another difference from the first embodiment, in the first embodiment, the light intensity detection unit 28 includes the photodiode (Red) 38, the photodiode (Green) 39, and the photodiode (Blue) 40 having different spectral sensitivity characteristics. In contrast, in the second embodiment, only a photodiode 60 is included. The photodiode 60 has a spectral sensitivity characteristic of 420 to 640 nm, which encompasses the wavelength band of the xenon tube 3.

FIG. 7 is a configuration diagram of electrical circuits of the xenon tube drive circuit 24 and the light intensity detection unit 28 according to the second embodiment. As the difference from the first embodiment, the first embodiment employs the configuration in which the light intensity detection unit 28 includes three photodiodes and hence three circuits having the same configuration, but in this embodiment, only one photodiode 60 is included to reduce the number of circuits to one.

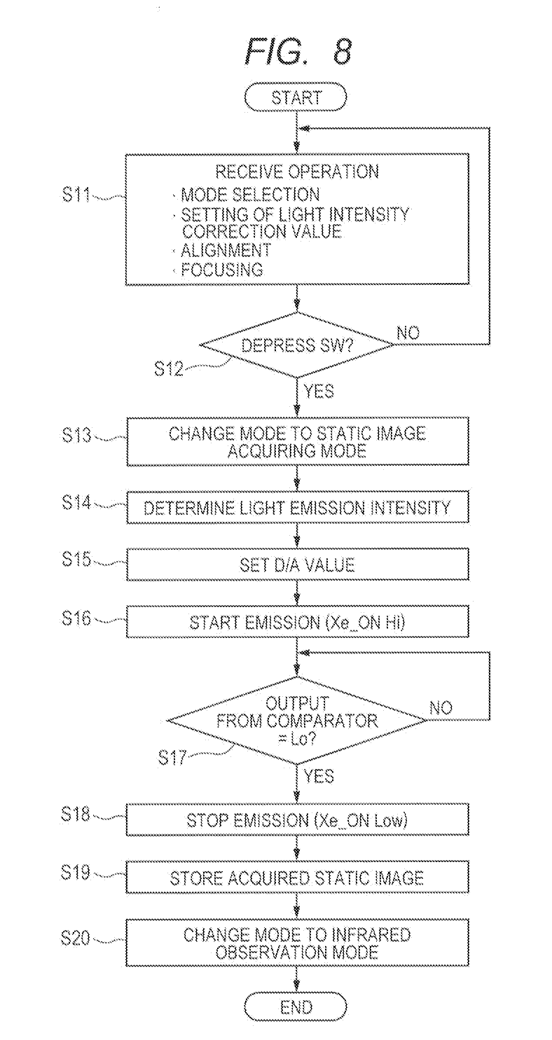

An imaging sequence according to the second embodiment is described with reference to a flowchart of FIG. 8.

In Step S11, the operator selects the color imaging or the filter imaging. When the color imaging is selected, the band pass filter 59 and the band pass filter 61 for the filter imaging are retracted from the optical axis. After the alignment and the like are finished, the operator turns on the imaging switch (Step S12). At the time when the processing proceeds to Step S13, the analog switch 50 of the light intensity detection unit 28 is in an ON state, and the integrating capacitor 44 is in a reset state.

In Step S13, the mode is changed from the infrared observation mode to the static image acquiring mode.

In Step S14, the light emission intensity is calculated based on the set imaging mode and light intensity correction value.

In Step S15, based on the determined light intensity, the standard D/A value is determined with the light intensity table, and the CPU 29 passes the standard D/A value to the D/A converter 53. The D/A converter 53 outputs the standard voltage to the comparator 56.

In Step S16, the CPU 29 turns off the analog switch 50 to cancel the resetting of the integrating circuit. Thereafter, the emission is started.

In Step S17, in both of the color imaging and the filter imaging, the CPU 29 first waits until the output of the comparator 56 becomes Lo, and when the output becomes Lo, the processing proceeds to Step S18. As opposed to the first embodiment, during the filter imaging, the band pass filter 61 is placed between the xenon tube 3 and the stop 27, and hence the light having the same wavelength as the light that illuminates the fundus enters the photodiode 60.

In Step S18, the emission is stopped.

In Step S19, the static image is stored.

In Step S20, the mode is changed to the infrared observation mode.

In this embodiment, the band pass filter and the driving mechanism therefor are added to make the configuration complicated, but the same effects as the first embodiment may be obtained.

### Third Embodiment

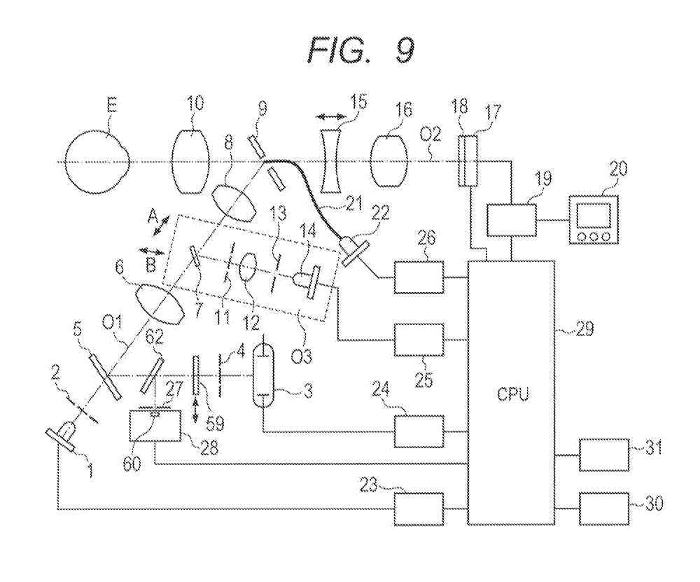

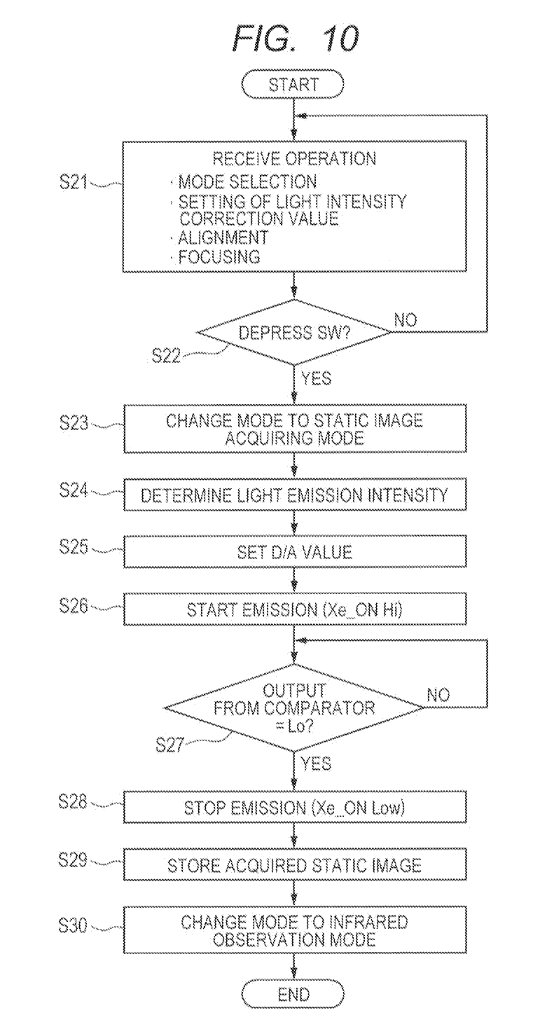

The present invention is described by way of a third embodiment illustrated in FIGS. 9 and 10.

The detailed description is given only of differences from the first and second embodiments.

FIG. 9 is a configuration diagram of a fundus camera according to the third embodiment. As with the second embodiment, the light intensity detection unit 28 includes the photodiode 60 having the spectral sensitivity characteristic of 420 to 640 nm. As a difference from the other embodiments, a half mirror 62 is placed between the band pass filter 59 and the dichroic mirror 5. In addition, the light intensity detection unit 28, which is placed behind the xenon tube 3 when viewed from the fundus illumination optical system O1 in the other embodiments, is placed in a reflection direction of light extracted from a principal light beam by the half mirror 62. The reflected light from the half mirror 62 is configured so that a part thereof enters the photodiode 60 through the stop 27, which is placed between the half mirror 62 and the light intensity detection unit 28. In other words, the half mirror 62 functions as a unit placed between the band pass filter 59 as the imaging light wavelength selection unit and the fundus to extract a part of the light that illuminates the fundus, and the photodiode 60 as the light intensity detection portion uses the extracted light to detect the emission intensity.

In this embodiment, the configurations of the electrical circuits of the xenon tube drive circuit 24 and the light intensity detection unit 28, and their input/output relationships with the CPU 29 and the xenon tube 3 are the same as in the second embodiment.

An imaging sequence according to the third embodiment is described with reference to a flowchart of FIG. 10.

In Step S21, the operator first selects the color imaging or the filter imaging with the operation portion. When the color imaging is selected, the band pass filter 59 for the filter imaging is retracted from the optical axis. After the alignment and the like are finished, the operator turns on the imaging switch (Step S22).

In Step S23, the mode is changed from the infrared observation mode to the static image acquiring mode.

Further in Step S24, the light emission intensity is calculated.

In Step S25, the standard voltage is set to the comparator 56.

In Step S26, the emission is started.

In Step S27, in both of the color imaging and the filter imaging, the CPU 29 first waits until the output of the comparator 56 becomes Lo, and when the output becomes Lo, the processing proceeds to Step S28. As opposed to the other embodiments, in the filter imaging, the imaging light that has passed through the band pass filter 59, which is placed on the optical axis, enters the photodiode 60.

In Step S28, the emission is stopped.

In Step S29, the static image is stored.

In Step S30, the mode is changed to the infrared observation mode.

In this embodiment, the half mirror is added and a part of the imaging light needs to be extracted for detecting the light intensity, but the same effects as the first embodiment may be obtained.

As described above, according to the present invention, the control error of the imaging light due to the individual difference in emission wavelength distribution of the imaging light sources may be suppressed by switching the wavelength to be monitored to the wavelength band equivalent to the light that actually illuminates the fundus depending on the imaging mode. Further, the control does not use the reflected light, and hence is not affected by the ghosts contained in the reflected light. There may be provided an apparatus that has a plurality of imaging modes having different wavelengths for illuminating the fundus, and is capable of controlling, in each of the imaging modes, the light for illuminating the fundus to have a desired light intensity at high accuracy.

### Other Embodiments

Further, the present invention is also implemented by executing the following processing. Specifically, in this processing, software (program) for implementing the functions of the above-mentioned embodiments is supplied to a system or an apparatus via a network or various kinds of storage medium, and a computer (or CPU, MPU, etc.) of the system or the apparatus reads and executes the program.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2012-195299, filed Sep. 5, 2012, which is hereby incorporated by reference herein in its entirety.

What is claimed is:

1. An ophthalmologic imaging apparatus, comprising:
an imaging light source for illuminating a fundus;
a light intensity detection unit for monitoring, at a time of imaging, an emission intensity from a start of emission of the imaging light source;
an imaging light wavelength selection unit for selecting a wavelength band of light that is emitted from the imaging light source and illuminates the fundus; and
a detection wavelength changing unit for changing a wavelength band of light to be guided from the imaging light source to the light intensity detection unit depending on the wavelength band selected by the imaging light wavelength selection unit.

2. An ophthalmologic imaging apparatus according to claim 1, wherein the light intensity detection unit includes a plurality of light intensity detection portions having different detection wavelengths, and the detection wavelength changing unit switches at least one of the plurality of light intensity detection portions for use depending on the selected wavelength band.

3. An ophthalmologic imaging apparatus according to claim 1, wherein the detection wavelength changing unit includes a band pass filter for detection unit provided between the imaging light source and the light intensity detection unit, and the band pass filter for detection unit is inserted to and removed from an optical path between the imaging light source and the light intensity detection unit depending on the selected wavelength band.

4. An ophthalmologic imaging apparatus according to claim 1, wherein the detection wavelength changing unit includes a unit placed between the imaging light wavelength selection unit and the fundus to extract a part of the light that illuminates the fundus, and the light intensity detection unit uses the extracted part of the light to detect the emission intensity.

5. An ophthalmologic imaging apparatus according to claim 1, wherein the light intensity detection unit monitors light that is emitted from the imaging light source without the light passing through the fundus.

6. An ophthalmologic imaging apparatus according to claim 2, further comprising a control unit for controlling others of the plurality of light intensity detection portions that are not selected by the detection wavelength changing unit to output a predetermined signal in monitoring the light emitted from the imaging light source.

7. An ophthalmologic imaging apparatus according to claim 2, wherein the light intensity detection unit monitors the emission intensity of the imaging light source based on a standard signal depending on an emission intensity corresponding to the wavelength band of the light that illuminates the fundus, which is selected by the imaging light wavelength selection unit.

8. An ophthalmologic imaging method, comprising:
selecting light having a predetermined wavelength band from light emitted from an imaging light source and illuminates a fundus;
determining an emission intensity of the light having the predetermined wavelength band at a time of imaging;
monitoring light that has the predetermined wavelength band and has not passed through the fundus; and
stopping emission of the imaging light source depending on the monitoring of the light.
